(19) United States
(12) Patent Application Publication
(10) Pub. No.: US 2018/0236166 A1
(43) Pub. Date: Aug. 23, 2018

(54) DETECTION AND INTERRUPTION DEVICES FOR INFUSION LEAKAGE AND THE MONITORING SYSTEM THEREOF

(71) Applicant: Spectronik Medical Inc., Taipei (TW)

(72) Inventors: Yin Chang, Taipei (TW); Chiao-Li Lin, Taipei (TW); Daow-Ming Jiang, Taipei (TW)

(21) Appl. No.: 15/903,750

(22) Filed: Feb. 23, 2018

(30) Foreign Application Priority Data
Feb. 23, 2017 (TW) 106106220

(51) Int. Cl. A61M 5/168; A61M 5/50; A61M 25/00

(52) U.S. Cl. CPC A61M 5/16836; A61M 5/5086; A61M 5/16877; A61M 25/0097; A61M 2205/15; A61M 2205/3313; A61M 2205/3592; A61M 2205/8206; A61M 2230/63; A61M 2205/18; A61M 2205/581; A61M 2205/583; A61M 2205/3561; A61M 2205/50

(57) ABSTRACT

An infusion-leakage detection device includes: a substrate; a circuit with an infusion-leakage detection region formed on the substrate and aligned to an IV catheter which is inserted into a blood vessel of a patient, and the region includes at least a light-emitting element emitting a light with wavelength within the second optical window of biological organization and at least a light detector receiving the light to generate an electrical signal; and a circuit and battery region formed on the substrate, including a control and calculation unit connecting to the light-emitting element, and an acceleration detector connecting to the control and calculation unit, sensing body movement of a patient and providing a body-movement signal to the control and calculation unit. A remote monitoring system including the infusion-leakage detection device and remote equipment which receive information of leakage/no-leakage and alarm signal to monitor infusion-leakage status remotely.

### FIELD OF THE INVENTION

The present invention relates to detection and interruption devices for infusion leakage during IV therapy and the monitoring system thereof, in particular, relates to an infusion-leakage detection device, an infusion interruption device and an infusion-leakage monitoring system.

### BACKGROUND OF THE INVENTION

When a light emits into a biological organization, it can be partially absorbed, reflected, scattered or can partially penetrate the organization, and the absorbing feature can be presented by a coefficient μ_{α}(cm⁻¹), and the reciprocal of the coefficient is defined as the propagation depth of the light penetrating into an absorbing medium (mean free path). The scattering of photons in the organization decides the distribution of a 3-D volume of light intensity thereof. Scattered photons are simply changed in the path but not losing the energy. Scattering coefficient can be represented by μₛ(cm⁻¹), and the reciprocal of the coefficient represents an average free path from the present scattering point to the next one. Isotropy is not the feature of light-scattering in a biological organization. Instead, forward scattering occupies higher proportion of light-scattering in a biological organization. Such feature can be represented by anisotropy “g”, and g is an absolute value from 0 (isotropy) to 1 (fully forward scattering). In a biological organization, the value of g is usually between 0.7 and 0.95. When practical scattering conditions are considered by the g value, the original scattering coefficient attenuates to μₛ'(cm⁻¹) wherein μ_{x′}=μₛ(1-g), and the sum of μₛ and β_{α}is called total attenuation coefficient μₜ(cm⁻¹): βₜ=μₛ+βα.

The energy-transferring in an organization can be described by transport theory (referring to Chandrasekhar S., Radiative Transfer. New York, N.Y., Dover Publications Inc. 1960.), represented by the formula below:

s·∇L(r, s)=−(μ_{α}μₛ)L(r, s)+μₛ∫_{4π}p(s, s′)L(r, s′)dω′

The formula states that the intensity of the radiance L(r, s) located at a point “r” with a unit directional vector “s” in a space may decrease resulting from the absorbing and scattering by the medium when the light emits into the medium. Sometimes the radiance intensity may increase resulting from other light scattered from another directional vector s' so that the intensities are added up. The light radiance describes that the amount of light that travels through a particular area or from a particular area and falls within a specified solid angle. Where d⋅′ is the difference in solid angle in the s′ direction, and p (s, s′) is the phase function. Since μ_{α}, μₛ and p (s, s′) are required to calculate the distribution of light according to the above formula in the biological organization, and these parameters are not fixed in a biological tissue [heterogeneity], it is indeed a considerable difficulty to calculate the distribution of the radiance. The Monte Carlo method is a computational method that relies on repeated random sampling to obtain numerical results. The basic idea is to use randomness to solve problems that may have been identified in principle. When physics and mathematics problems are hard to solve or cannot find other available methods, it can be the most useful way to reasonably solve the problem mentioned above by simulation.

Monte Carlo simulation has been used by many people in the behavioral analysis of photon absorption and diffusion in different tissues [Wilson B C, Adam G (1983) A Monte Carlo model for the absorption and flux distributions of light in tissue. Med Phys 10:824-830.]. In addition, the article “Monte Carlo simulation of photon migration in tissue” [Chapter 2, in “Application of Near Infrared Spectroscopy in Biomedicine”. Springer, ISBN: 978-1-4614-6251-4] describes the simulation of photon migration in different thickness layers in fat tissue. When the distance between the light-emitting element and the light detector is fixed, and the thickness of tissue is varied, the number of the photons moving to the detector is different. According to this, suppose that the thickness of the tissue is also fixed, then the number of the photons moves to the detector is nearly constant so that the output signal of the detector is also nearly constant. However, when a substance (such as the leaking liquid during IV therapy) infiltrates on the path of the photon migration between the light source and the detector, the detector's signal decreases.

The main content within the liquid medicine for IV therapy is water. FIG. 1A is an absorption spectrum of water in a specific wavelength range. If a light-emitting element having a proper wavelength is chosen, the detecting unit apart at a proper distance from the light-emitting element should receive the light emitted from the light-emitting element. If a liquid (water may be the principal composition) exists on the path between the emitting end and the receiving end (including the receiving end and the emitting end) of the light-emitting and the detecting elements, the light can be absorbed by the liquid and resulting in a reduced signal at the light-detecting unit, and the quantity of the reduction should be proportional to the amount of the leaking liquid.

Currently, there are some examples applying such optic technology for detecting infusion leakage. In patent U.S. Pat. No. 7,826,890 a package of a light-emitting device surrounded by four light-receiving devices was designed for infiltration detection (FIG. 6 of U.S. Pat. No. 7,826,890). In U.S. Pat. No. 6,487,428, a complicated package including four emitting devices and eight receiving devices is disclosed (FIG. 2 of U.S. Pat. No. 6,487,428). Moreover, in CN 103596608 a light-emitting-receiving device including four light-emitting devices and a receiving device is used to detect infusion leakage (FIG. 4 of CN 103596608). No matter which kind of package in the above art is, all of these infusion-leakage detecting devices or equipment have several main problems, which they are difficult for the operator (usually a nurse) to align the detecting device to the IV catheter at a proper position so that the detecting element may not have higher output signal and could miss infusion-leakage event. Additionally, the wires (optic fibers or electrical wires) connected to the proximal sensing and distal signal processing parts of the device become a problem to obstacle the movement of the limb. Also these wires could affect or spur the sensing device that may results in a disturbance of signal collection and faulty message of infusion-leakage. The extended problem which can be seriously affect a correct leakage/no-leakage signal collection is the body movement if no adequate mechanism can be applied to eliminate the error signal of the body movement.

In addition, in column 5, lines 49-60 of U.S. Pat. No. 7,826,890, it emphasizes that a light having 850 nm wavelength may penetrate deeper organization, and the light having such wavelength is not easily absorbed by water and ordinary pigments. The application of the “first biological window” of optics for biological organization is used, and the wavelength range of the first biological window is between 650 and 950 nm. The definition of the so-called biological window of a biologic organization is that a light may more easily penetrate the organization under the wavelength range (i.e. less absorbed by the organization). However the paper “The Complex Refractive Index of Water” by D. J. Segelstein (MS thesis, University of Missouri, 1981) and the paper “Near-infrared spectroscopy as an index of brain and tissue oxygenation” Br. J. Anaesth. (2009) 103 (suppl 1): i3-i13 doi:10.1093/bja/aep299, indicated that light has less absorptivity in water below 850 nm than those above it, as shown in FIG. 1A, but the absorption rate of melanin is reversed, as shown in FIG. 1B (FIG. 1 of the paper “Near-infrared spectroscopy as an index of brain and tissue oxygenation” British Journal of Anaesthesia, 103 (BJA/PGA Supplement), i3-i13 (2009) doi:10.1093/bja/aep299, by J. M. Murkin and M. Arango). Meanwhile, the FIG. 1 of the paper “Bioimaging: second window for in vivo imaging” Nat Nanotechnol. 2009 November; 4(11): 710-711.doi: 10.1038/nnano.2009.326 disclosed by A. M. Smith, M. C. Mancini, and S. Nie), indicates that there is a second window having wavelength between 1000-1350 nm, as shown in FIG. 1C of the present invention. In other words, the light with wavelength within the second optical window has much lower absorptivity for melanin which implies that the light with wavelength within the second window can penetrate skin deeper than those in the first optical window.

Accordingly, a design for infusion-leakage detection that contains a light-emitting and light-detecting elements having specific wavelengths within or covering the second biological window should achieve the aim of infusion-leakage detection. If the technology of wireless data communication can be applied in this task, the spurred-wire problem mentioned above can be avoided. Again, the body movement sensing and the body movement error signal eliminating is a key factor to have a successful and accurate infusion-leakage detection. The extended functions of this design are that the signal of infusion-leakage can be in real time remotely monitored, the infusion conduit (tubing) can be automatically blocked by an interruption device when leakage is sensed by the device.

### SUMMARY OF THE INVENTION

To achieve aforesaid objects, the present application provides an infusion-leakage detection device, an infusion-leakage interruption device and an alarm and remote monitoring system. The present application can not only detect infusion-leakage during IV therapy, also and it can interrupt the flow of the infusion conduit in time to stop extending tissue damage when leakage occurs. The status signal of infusion leakage/no-leakage in patient is sent to the computer of nurse station and to the smart phone/tablet of the on-duty nurses or physicians at any time. Once infusion leakage is detected, warning signals, such as flashing LED and buzzer alarm on the device are activated. In the meantime, message of leakage with warning signal is also sent to the smart phone/tablet and the computer of the nurse station simultaneously, and the infusion interruption device is activated too.

The present application has composite features, in addition to the functions of infusion-leakage detection and alarm, it also includes the sensing part of the infusion-leakage detection device for aligning the sensing region of the device to the IV catheter in order to obtain a better sensitivity, small size, convenience for use, immunity of the actual leakage/no-leakage signal from body movement, remote monitoring patient's current status of leakage/no-leakage with smart phone/tablet at a remote site by on-duty nurses or physicians, and computer at the nurse station.

The present application provides an infusion-leakage detection device including: a substrate; a circuit and infusion-leakage detection region formed on the substrate at can hemi-surround the hub at the proximal end of an IV catheter and be aligned to the IV catheter which has been inserted into a blood vessel of a patient, and the region includes at least a light-emitting element and at least a light detector, wherein the light-emitting element emits a light with wavelength range within or covering the second optical window of biological organization to a target organization of a human body, and the light detector receives the light reflected, transmitted, diffused or scattered from the target organization to generate an electrical signal; and a circuit and battery region formed on the substrate, comprising a control and calculation unit and an acceleration detector wired to the control and calculation unit; wherein the control and calculation unit connects to the light-emitting element to control the light intensity of the light-emitting element; wherein the acceleration detector senses body movement of a patient and provides a three dimensional acceleration signal of the movement to the control and calculation unit, and the control and calculation unit judges whether the body movement influences the actual infusion-leakage signal.

The present application also provides an infusion-leakage detection device, including: the infusion-leakage detection device; a server; and an infusion-leakage interruption device: wherein the infusion-leakage detection device transfers the detected data and alarm signal to the server and infusion interruption device via a wireless technique or network; wherein the server connects to an intranet network system of a hospital so that the detected infusion-leakage data and warning signal can be delivered to the smart phones of on-duty nurses/physicians and a computer of the hospital; wherein the infusion-leakage interruption device is activated when receiving the signal from the infusion-leakage detection device to block the infusion flow of the infusion conduit to cease the leakage not to get worse.

The infusion-leakage detection/interruption devices can further include a signal processing circuit and a battery with power management circuit on the substrate, wherein the signal processing circuit contains an amplifier for amplifying the infusion leakage/no-leakage signal coming from the light detectors. The control and calculation unit is for converting the analog signal output from the amplifier to digital signal and making leakage/no-leakage decision by an embedded algorithm. A communication unit receives an alarm signal from the control and calculation unit and sends patient's ID code and the alarm signal to the remote monitoring equipment and the leakage-interruption device if leakage occurs, and receives the acknowledgement signal from the leakage-interruption device as a confirmation of activation. The battery with power management circuit contains a button battery and related circuit which distributes corresponding regulated voltage to targeted electrical components and circuits.

The present application also provides a remote monitoring system, including wireless gateway, server and mobile equipment (smart phone/tablet) and the computer at the nurse station. The status signals of leakage/no-leakage in patients are sent to the mobile equipment and the computer at the nurse station by the infusion-leakage detection device periodically through the wireless network. When leakage occurs, the mobile equipment and the computer at the nurse station alarm, also the patient's bed is also highlighted shown on the screen. The advantage of this design is that the nurse needs not to check the patients periodically and can do the other work at the saved time.

To make the aforesaid and other objects, features and advantages of the present invention can be more apparent and easier to be understood, some embodiments are introduced in the following quotes, and in together with the accompanying drawings to make a detailed description below (embodiments).

### BRIEF DESCRIPTION OF THE DRAWINGS

FIG. 1A shows an absorbing the absorption spectrum of water in a specific wavelength range. (D. J. Segelstein, “The complex refractive index of water,” University of Missouri-Kansas City (1981)).

FIG. 1B shows an absorbing the absorption spectrum of HbO₂, Hb, melanin and cytochrome oxidase (Caa3) in a specific wavelength range. The vertical axis is absorption. (FIG. 1 of the paper “Near-infrared spectroscopy as an index of brain and tissue oxygenation” British Journal of Anaesthesia, 103 (BJA/PGA Supplement), i3-i13 (2009) doi:10.1093/bja/aep299, by J. M. Murkin and M. Arango)

FIG. 1C shows an absorbing the absorption spectrum of HbO₂, Hb, skin and fat in a specific wavelength range. (FIG. 1 of the paper “Bioimaging: second window for in vivo imaging” Natural Nanotechnology. November; 4(11): 710-711 (2009).doi:10.1038/nnano.2009.326, by A. M. Smith, M. C. Mancini and S. Nie)

FIG. 2A illustrates a schematic diagram of a detection and interruption devices for infusion-leakage and the network of remote monitoring system thereof of an embodiment of the present application.

FIG. 2B illustrates a schematic diagram of a detection and interruption devices for infusion-leakage and the network of remote monitoring system thereof of another embodiment of the present application.

FIG. 3A illustrates a functional block diagram of an infusion-leakage detection and interruption device of the present application.

FIGS. 3B and 3C illustrate a schematic functional block diagram and a schematic operation diagram of the present application for infusion-interruption device, respectively.

FIG. 4A illustrates a schematic arrangement diagram of the detection device for infusion-leakage of an embodiment of the present application.

FIG. 4B illustrates a schematic arrangement diagram of the detection device for infusion-leakage of another embodiment of the present application.

FIG. 4C illustrates a schematic arrangement diagram of the detection device for infusion-leakage of another embodiment of the present application.

FIG. 4D illustrates a schematic arrangement diagram of the detection device for infusion-leakage of another embodiment of the present application.

FIG. 5 illustrates a simulation diagram for the infusion-leakage signal of the infusion-leakage detection device of the present application.

FIG. 6 illustrates a typical result of an animal experiment for infusion-leakage detection of the present application.

### DETAILED DESCRIPTION OF THE INVENTION DEFINITIONS

According to an embodiment of the present application, a system including infusion-leakage detection device, infusion interruption device and wireless remote monitoring network is disclosed. The present application can be applied to every peripheral intravenous (PIV) infusion process of medical treatment, therefore patients can obtain the benefits from this technique including real time monitoring PIV infusion-leakage with any kind of situation, errors-prevention resulted from body movement, infusion interruption when leakage occurs, and wirelessly remote monitoring the status of leakage/no-leakage during IV therapy.

Pairing is a wireless network technique to connect two devices each other by a standard procedure. Pairing for patient and the infusion-leakage detection system can be pre-processed before the infusion-leakage detection device being put on patient's body. This process can be done at the patient's bedside with dedicated smart phone/tablet or at the remote computer site at the nurse station.

After pairing is done, the infusion-leakage detection device is then put on the patient's body where the infusion takes place. The entire system is activated after the IV catheter being inserted into the blood vessel and a “function” button being pressed on the device. At this time the device starts to do auto-calibration, and the LED of the device flashes yellow light for several second when the calibration is done. Then the status of the device such as battery power, and the information of leakage/no-leakage is transmitted to the remote nurse station and the smart phone/tablet of the on-duty nurses and physicians. If leakage occurs, the LED and the buzzer on the device will flash light and tweet as alarm signals. Meanwhile this warning message is also passed to the nurse station and the on-duty nurse's/physician's smart phone/tablet too.

The wavelength range of the light source applied to the present application can be between 1000 nm and 1350 nm or little wider, which is within or covers the second optical window of biological organization. Thus the light chosen within this window that absorbed by melanin,

HbO2 and Hb shall much less than most of lights having wavelengths within the first optical window of biological organization but highly absorbed by water, therefore the wavelengths chosen within the second optical window shall fully meet the demand of infusion-leakage detection of the present application and has better sensitivity than those outside this optical window.

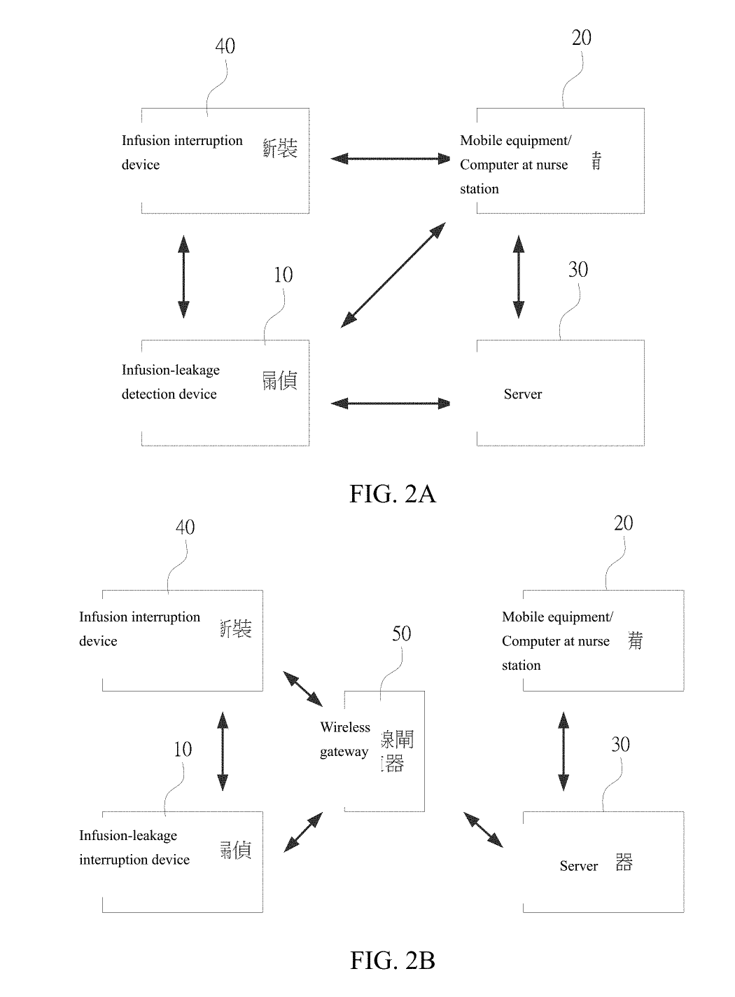

FIG. 2A illustrates a schematic diagram of infusion-leakage detection and interruption devices and remote monitoring system of an embodiment of the present application. The infusion-leakage detection and interruption devices and the monitoring system of the present application include: an infusion-leakage detection device 10, mobile equipment 20, server 30 and infusion interruption device 40. The infusion-leakage detection device 10 transfers the detected data (leakage/no-leakage) and alarm signal (if there is leakage detected) to the mobile equipment 20, server 30 and infusion interruption device 40 via a wireless technique or network. Wherein the server 30 can connect to the hospital's intranet network system so that the detected infusion-leakage data and warning signal (if any) can be delivered to the computer at the nurse station and the mobile equipment of the on-duty nurses and physicians. The infusion interruption device 40 is activated at the same time, when infusion leakage is detected, and the flow of infusion is then blocked in the infusion conduit to cease the leakage, and an acknowledgement signal is sent to the mobile equipment/nurse station and the detection device 10. Accordingly, the infusion-leakage detection device 10, mobile equipment/nurse station 20, server 30 and infusion interruption device 40 can communicate each other wirelessly.

FIG. 2B illustrates a schematic diagram of infusion-leakage detection and infusion interruption devices and the remote monitoring system thereof of another embodiment of the present application. The infusion-leakage detection and infusion interruption devices and the remote monitoring system of the present application includes: an infusion-leakage detection device 10, mobile equipment/nurse station 20, server 30, infusion interruption device 40 and a wireless gateway 50. Any two of the above devices, shown by double head arrow, can communicate each other wirelessly. The infusion-leakage detection device 10 transfers a detected infusion leakage/no-leakage signal and alarm signal (if leakage is detected) to the wireless gateway 50, and the wireless gateway 50 transfers the data to the server 30 via wireless LAN. Wherein the server 30 can connect to mobile equipment/nurse station 20 to log the leakage/no-leakage data, and gives a warning signal if leakage happens. Of course same network pathway can also apply to the infusion interruption device 40 too. The infusion-leakage detection device 10 can communicate with the other parts each other (same as FIG. 2A), bypassing the wireless gateway 50 if gateway 50 does not function for some unknown reasons, so do the mobile equipment/nurse station 20 and the server 30.

When interruption is activated, the interruption device 40 will immediately responds an acknowledgment signal to the mobile equipment/nurse station 20 and the detection device 10 by way of wireless gateway 50 and sever 30.

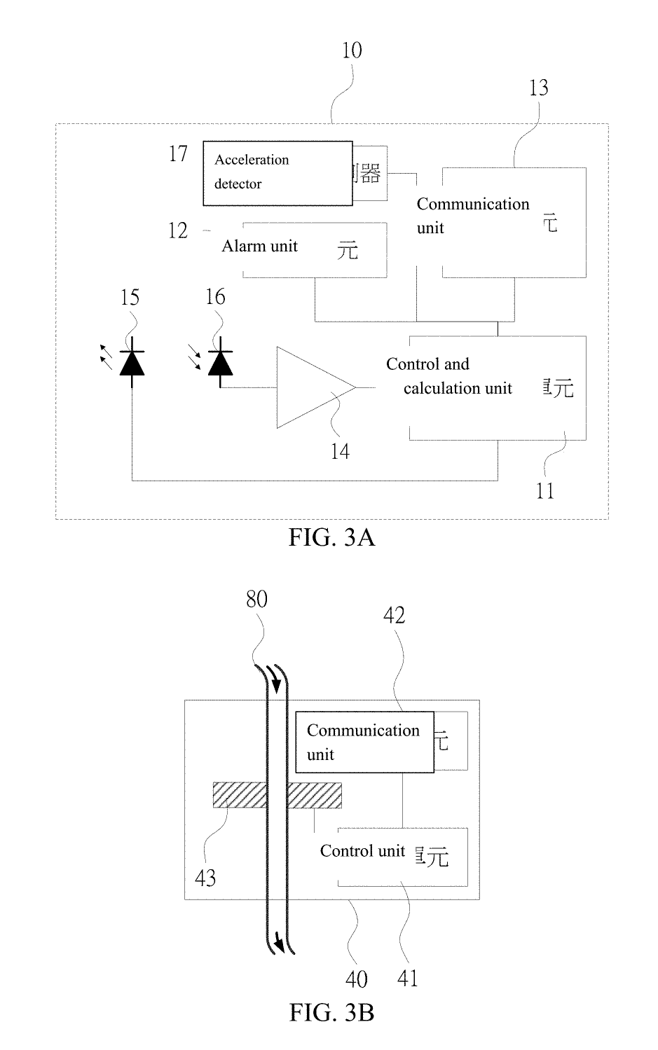

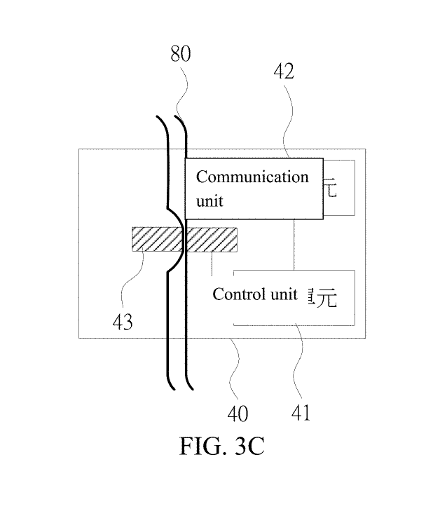

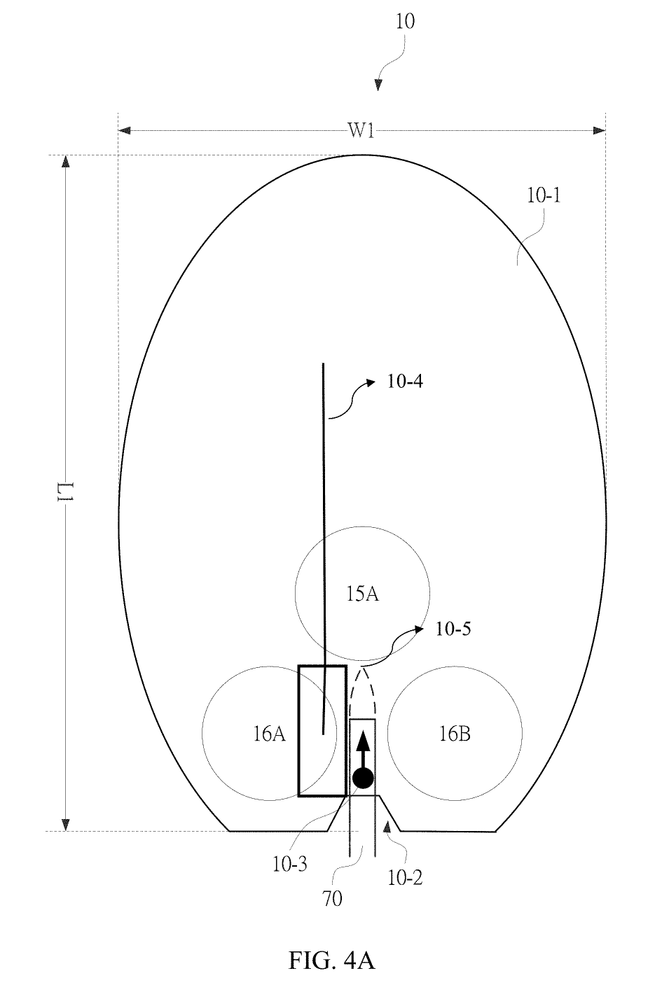

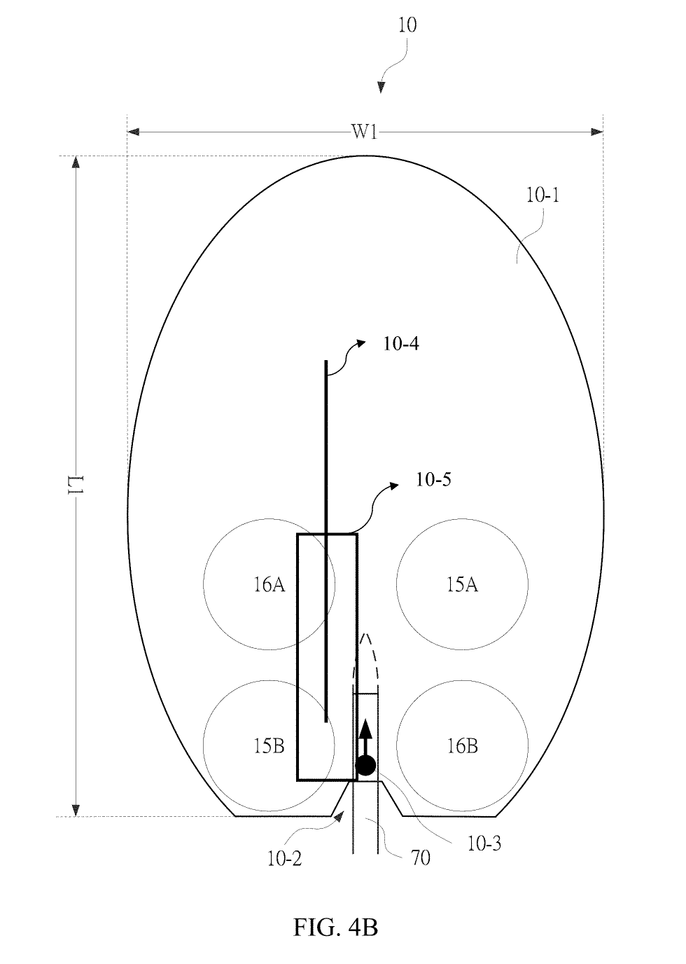

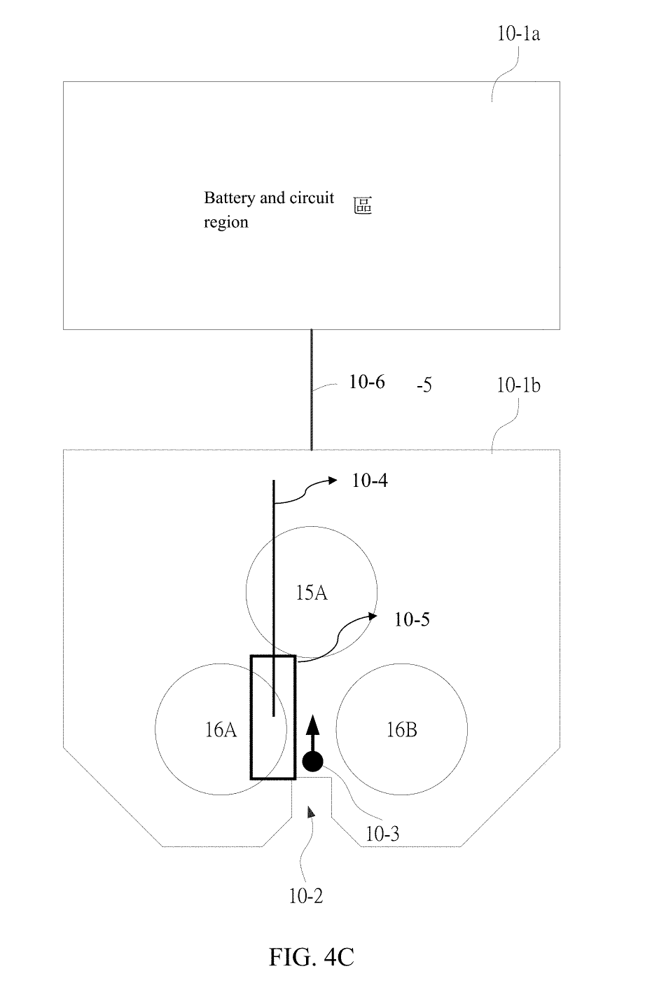

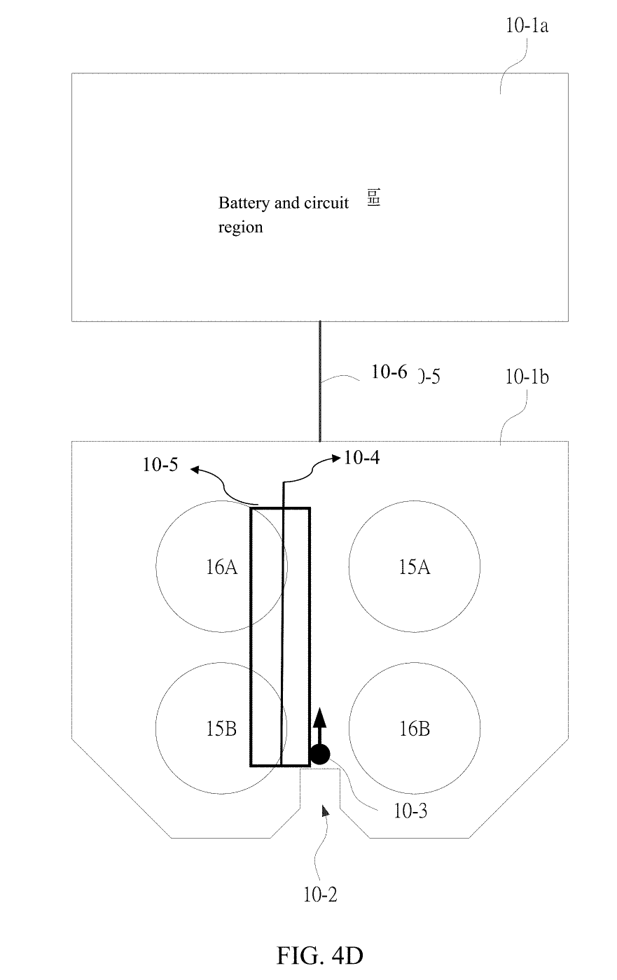

FIG. 3A illustrates a functional block diagram of an infusion-leakage detection device 10 of the present application. The infusion-leakage detection device 10 includes: an infusion-leakage detecting device, an amplifier 14, a control and calculation unit 11, an alarm unit 12, a communication unit 13 and an acceleration detector 17. The infusion-leakage detection device includes: a light-emitting element 15 and a light detector 16 (the number thereof are at least two, and the specific arrangement is shown in FIGS. 4A and 4B), wherein the light-emitting element 15 emits a light with wavelength within or covering the second optical window of biological organization to a target organization of a human body, and the light detector 16 receives the light reflected, transmitted, diffused or scattered from the target organization to generate a received electrical signal. The amplifier 14 receives and amplifies the signal from the light detector 16 and feeds this signal forward to the control and calculation unit 11 for further data processing. The alarm unit 12 is for generating alarm signals of LED flashing light and buzzer tweeting sound on the device 10 itself when the detected signal is unmet the preset threshold after the judgement by an algorithm. The control and calculation unit 11 connects the amplifier 14, light-emitting element 15, alarm unit 12, communication unit 13 and acceleration detector 17 to control the intensity of light emitted from the light-emitting element 15, and make decision about whether an alarm command has to be announced to the alarm unit and the communication unit after the judgement by the algorithm embedded in this control and calculation unit. The alarm unit generates alarm signals when receives alarm command from the control and calculation unit 11. The control and calculation unit 11 also send leakage/no-leakage signal to the communication unit 13 by which the signals can be delivered to the remote monitoring equipment and the infusion interruption device. The light emitted from the light-emitting elements 15 can be a DC light or an AC (sinusoidal or pulsed) light.

The light intensity of the light-emitting element 15 can be controlled by the control and calculation unit 11. The light received by the light detector 16 is converted to an electrical signal and amplified by the amplifier 14 (for example, multiple stages of amplifying and filtering), and then converted to a digital signal via an analog to digital converter (ADC) of the control and calculation unit 11. This digital signal is then processed and judged by an algorism, which is embedded in this unit 11. The judged signal is transmitted to the mobile equipment/nurse station 20 through the communication unit 13, or to the wireless gateway in FIG. 2B through the communication unit 13, and reach to the mobile equipment/nurse station 20 finally. When a leakage occurs, the amount of light received by the light detector 16 decreases. If the leakage continued, the light received by the light detector 16 keeps on decreasing. The attenuation of the light signal is proportional to the volume of the leaked infusion liquid. When the attenuation of the light signal exceeds a leakage threshold value determined by the algorithm, the control and calculation unit 11 sends alarm command to the alarm unit 12 that will activate LED to flash and buzzer to tweet, and to the mobile equipment/nurse station 20 by way of the communication unit 13 via the framework of FIGS. 2A or 2B. Meanwhile, the infusion interruption device 40 is activated when leakage occurs.

Wherein the communication unit 13 bridges the control and calculation unit 11 and the external equipment. The external equipment can be the mobile equipment/nurse station 20, server 30 or the infusion interruption device 40 in FIG. 2A or the infusion interruption device 40 and the wireless gateway 50 in FIG. 2B. When the received electrical signal is judged as leakage after signal processing by an algorithm, the control and calculation unit 11 will inform the communication unit 13 to send a command to the infusion interruption device 40 and a warning signal to the mobile equipment/nurse station unit 30 directly or indirectly according to the frame work shown in FIG. 2A or FIG. 2B.

Wherein the acceleration detector 17 wired to the control and calculation unit 11 is to sense body movement. The acceleration detector 17 is applied in a situation: no matter whether the movement occurred or not of the patient's body, it provides a 3D acceleration signals to the control and calculation unit at any time. If there is any movement that influences the stability of the infusion-leakage detection device at the infusion site, this movement signal will be removed from the received infusion-leakage signal by signal processing of an algorithm in this unit 11. The sensitivity of the acceleration detector is adjustable through the control and calculation unit 11. Therefore, a judging mechanism of an algorithm is added to the present application. When the acceleration signal generated by the acceleration detector 17 and detected by the control and calculation unit 11 is over a threshold value of the acceleration, and causes the received leakage signal over the leakage threshold value, however, after removing the body movement signal and resulting in “no-leakage” by the judgement of the algorithm, the alarm signal will not be generated in the time duration of the body movement. Otherwise, if both infusion leakage and body movement occur in the meantime, “leakage” is announced and alarm command is sent out by the control and calculation unit 13.

FIGS. 3B and 3C illustrate a schematic block diagram of the infusion interruption device 40 and its operational diagram, respectively, of the present application. The infusion interruption device 40, shown in FIG. 3A, includes several elements: a control unit 41, a communication unit 42 and a pinch valve 43. In which the infusion conduit (or infusion tubing) 80 is placed in fixed holder of the pinch valve 43. Once the communication unit 42 receives the alarm command signal transmitted from the infusion-leakage detection device 10 or from the wireless gateway 50, it triggers the control unit 41 to control the pinch valve 43 to pinch off the infusion conduit 80 (as shown in FIG. 3C). An acknowledgement signal of completing the interruption is then transmitted to the infusion-leakage detection device 10, the mobile equipment and the computer of the nurse station. Meanwhile an LED on the infusion interruption device flashes.

FIGS. 4A, 4B, 4C and 4D reveal different arrangements of the framework of the light emitting-receiving unit and the framework of the substrate of the present application are disclosed.

FIG. 4A discloses the infusion-leakage detection device of the first embodiment of the present application. The infusion-leakage detection device 10 of the present application includes: a circuit substrate with length L1 thereof that can be 2-5 cm, and the width W1 thereof can be 2-3 cm. The substrate includes circuit and infusion-leakage detection region 10-1, and an indicating region 10-2 which includes an open groove for hemi-surrounding the hub 70 at the proximal end of the IV catheter, and a transparent or semi-transparent area 10-5 by which skin can be seen through the area. There is an arrow marker 10-3 printed along the central line 10-4 for aligning the device 10 to the IV catheter. The widths of the indicating region 10-2 and the area 10-5 are approximately the same as the width (or little larger) of the hub 70.

As shown in the embodiment of FIG. 4A, the shape of the open groove of the indicating region 10-2 is an inverted letter V or U, formed by cutting a portion of the substrate. The marker 10-3 and the central line 10-4 in the indicating region are convenient for a user to align the hub 70 by which the direction of the placed catheter in blood vessel can be estimated so that the device 10 can be suitably put on the patient's skin with better leakage-detection sensitivity.

In addition, the substrate can be formed by a flexible printed circuit (FPC) or a printed circuit board (PCB).

In the embodiment of FIG. 4A, a sensing unit with three light emitting and receiving elements is arranged, including a light-emitting element 15A and two light detectors 16A and 16B. The light-emitting element 15A and the light detectors 16A and 16B are arranged in a form of triangle as shown in the figure. The distances between 15A and 16A, 15A and 16B can be 11-20 mm, and 16A-16B can be 7-10 mm.

The light-emitting element 15A emit a light to a target organization of a human body, and the light detectors 16A and 16B receive the light transmitted, reflected or scattered from the target organization, or the light penetrating through the target organization, and an electrical signal is generated and amplified by an amplifier, and then delivered to the control and calculation unit 11. In the embodiment, the light emitted from the light-emitting elements 15A can be absorbed in various level that results from how serious of the leakage occurred since the reduction of light being received by the light detector is proportional to the amount of the leaking liquid within the tissue. The form of the light emitted from the light-emitting element 15A can be DC or any form of AC (such as sinusoidal or pulsed). The light emitting elements 15A can be either LED or laser diode.

In the embodiment of FIG. 4B, two light-emitting elements 15A and 15B and two light detectors 16A and 16B are arranged. The light-emitting elements 15A and 15B and the light detectors 16A and 16B are arranged in a form of rectangular or square. The two light-emitting elements are positioned diagonally and same to the light detectors. To place the device 10 shown in

FIG. 4B on patient's body is the same as the description for the device 10 shown in FIG. 4A. Wherein the distances between light-emitting element 15A (or 15B) and light detector 16A (or 16B) can be 7 mm-10 mm. The distance between the light detector 16A (or 16B) and light emitting element 15B (or 15A) can be 11-20 mm.

Wherein the light-emitting elements 15A and 15B emit lights to a target organization of a human body, and the light detectors 16A and 16B receive the light transmitted, reflected or scattered from the target organization, or the light penetrating through the target organization, and an electrical signal is generated and amplified by an amplifier, and then delivered to the control and calculation unit 11. In the embodiment, the light emitted from the light-emitting elements 15A and 15B can be absorbed in various level that results from how serious of the leakage occurred since the reduction of light being received by the light detector is proportional to the amount of the leaking liquid within the tissue. The form of the light emitted from the light-emitting elements 15A and 15B can be DC or any form of AC (such as sinusoidal or pulsatile). The light emitting elements 15A and 15B can be either LED or laser diode.

In the embodiment of FIG. 4C, similar to FIG. 4A, a unit with light-emitting and receiving is arranged by the same order. The difference is that in the embodiment of FIG. 4C two substrates are adopted to replace the substrate in FIG. 4A, that is, the original circuit and the infusion-leakage detection region 10-1 are separated into two parts: a portion of circuit and battery region 10-1a, and the other portion of circuit and infusion-leakage detection region 10-1b. To bridge the communication of the two portions is through the flat conductive wires 10-6. The flat conductive wires 10-6 can be part of the portion of circuit and infusion-leakage detection region 10-1b. In other words, the flat conductive wires 10-6 can be either a part of the portion of circuit and infusion-leakage detection region 10-1b or can firmly connected to the 10-1b by way of a mini connector. At the other end, the flat conductive wires 10-5 is connected to the portion of circuit and battery region 10-1a also through a mini connector. The length of the flat conductive wires 10-6 can be variable so that the whole device 10 can fit various body locations with joint existed such as wrist, elbow and ankle.

In the embodiment of FIG. 4D, similar to FIG. 4B, a unit with four light emitting and receiving elements is arranged by the same order. The difference is that in the embodiment of FIG. 4D, two substrates are adopted similar to that of FIG. 4C, that is, the original circuit and the infusion-leakage detection region 10-1 are separated into two parts: a portion of circuit and battery region 10-1a, and a portion of circuit and infusion-leakage detection region 10-1b. To bridge the communication of the two portions is through the conductive wires 10-6. In other words, the flat conductive wires 10-6 can be either a part of the portion of circuit and infusion-leakage detection region 10-1b or firmly connected to it by way of a mini connector. At the other end, the flat conductive wires 10-6 is connected to the portion of circuit and battery region 10-1a through a mini connector. The length of the flat conductive wires 10-6 can be variable so that the whole device 10 can fit various body locations with joint existed such as wrist, elbow and ankle.

In the present application, because of the organization of tissue does not change during the period of the infusion therapy, the signal from the light-emitting element to the light detector is assumed to be a constant value (without body movement) since the distance between the light-emitting element and the light detector is fixed. Thus, the model of the present application can be simplified and explained by Beer-Lambert Law (Beer's Law). In a single biological structure, a light emitted from light-emitting element S through the organization having a thickness I, absorption coefficient α and medium concentration (or density) c to the light detector D. In accordance with Beer's Law: the relation is A=α1c. Wherein α is absorptivity, absorption coefficient or extinction coefficient.

The transmittance T of light is defined by a formula:

$$T = \frac{{I}_{e}}{{I}_{o}} ,$$

wherein I₀ is the light intensity of the light emitting into the organization, Iₑ is the light intensity after the light passing through the organization. The relation of the transmittance of light with the absorptivity is defined by a formula

$$A = - log T = - log ( \frac{{I}_{e}}{{I}_{o}} ) , or T = {10}^{- A} = {10}^{- α lc} .$$

If a light transmits a plurality of organizations having different thicknesses (I₁, I₂ . . . Iₙ) and the corresponding absorptivity and density of each organization is (α₁, α₂ . . . αₙ) and (c₁, c₂ . . . cₙ), respectively, and the total absorptivity Aₜ and the total transmittance Tₜ can be presented as equations (1) and (2), respectively.

Aₜ=α₁l₁c₁+α₂l₂c₂ + . . . + αₙlₙcₙ=A₁+A₂ + . . . + Aₙ (1)

Tₜ=T₁*T₂* . . . * Tₙ (2)

In above equations (1) and (2), it is assumed that the intrinsic tissue thickness of Iₙ (n=1, 2 . . . n) does not change when leakage occurs. If an infusion liquid such as water leaks, the total absorptivity A ₜ of the intrinsic tissue (Aₜ) and the leaked liquid (A*ₜ) can be added together and shown as equation (3).

A′ₜ=Aₜ+Aₜ^{*} (3)

wherein Aₜ^{*}=α_{H2O}* l_{H2O}*c_{H2O} (4)

After considering the leaking liquid, the transmittance become: T′ₜ₌*Tₜ*Tₜ^{*} (5)

Thus, when a situation of infusion-leakage is considered, the total transmittance Tₜ drops to Tₜ', and resulting in lowering the signal received by the light detector. The difference of the transmittance AT can be shown as equation (6)

ΔT=T′ₜ-Tₜ=10^{-A′}^{ₜ}-10^{-A}^{ₜ₌}10^{-A}^{ₜ}*10^{-A}^{ₜ}^{^{*}}-10^{-A}^{ₜ}=10^{-A}^{ₜ}* (10^{-A}^{ₜ}^{^{t−1)=}}k*(10^{-A}^{ₜ}^{^{*}}−1) (6)

wherein k represents the light signal detected by the detector before thaleakage occurs.

If simply considering the light signal received by the light detector, the signal value S_{λ}(function of wavelength) can be shown as equation (7)

S_{λ}=ε_{λ}T′ₜ=ε_{λ}Tₜ*Tₜ^{*}=ε_{λ}k*10^{-α}^{_{(H2O)}}^{^{l}}^{_{H2O(t)}}^{^{c}}^{_{H2O}} (7)

Where ε_{λ}represents the sensitivity of the light detector and is a function of wavelength. K=Tₜ, is the transmittance of light in the biological organization before that infusion-leakage occurs. α_{λ(H2O)} is the absorptivity of water when the wavelength of light is λ. When infusion-leakage continues, the l_{H2O}(t) increases with time (a function of time), and the total S drops, and c_{H2O} may also increase too.

FIG. 5 is the modeling of light received by the detector when leakage continuously occurs according to the mathematical derivation given above. Before the leakage being detected, the initial intensity of light detected by the detector is set as 1. When leakage occurs, the intensity of light detected by the detector decreases exponentially. The decreasing rate is proportional to the amount of the leakage. Thus the expected signal received by the infusion-leakage detection 10 can be as the pattern as shown in FIG. 5 when the infusion liquid is keeping leaking. The actually measured signal in practice may not be as smooth as the FIG. 5 does, so that when the control and calculation unit 11 in FIG. 3A samples the “leakage” signal, it may also contain the body movement signal by way of the acceleration detector (17), if there is any. The influence of the body-movement signal will be removed by the algorithm embedded in the control and calculation unit 11.

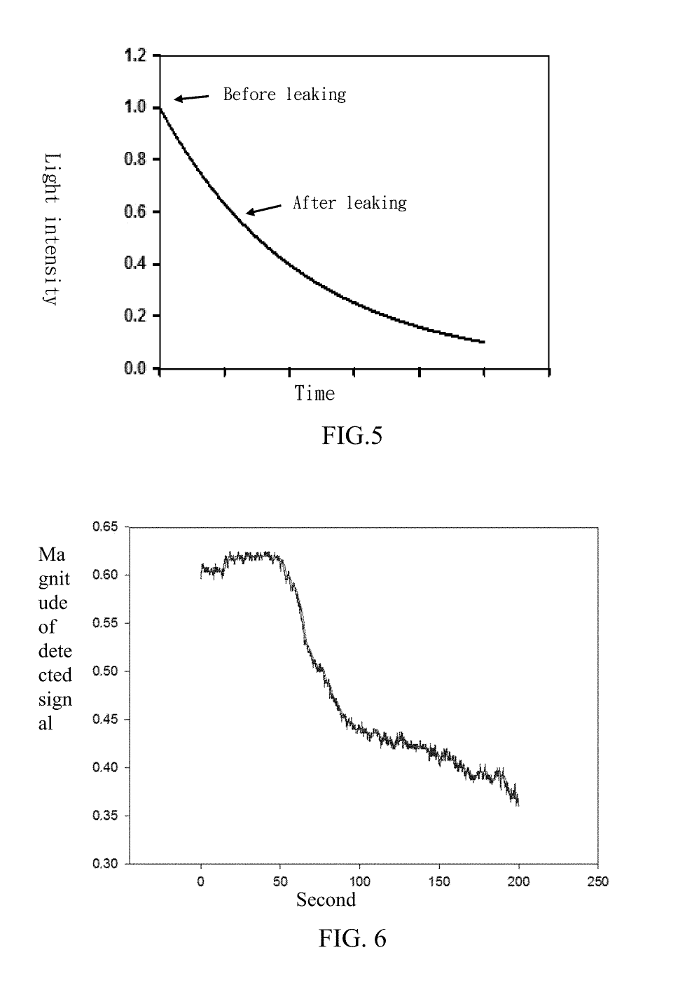

Please referring to FIG. 6, an experimental result on rats of the present application is disclosed. An IV catheter was inserted into the subcutaneous tissue of a rat leg (under anesthesia). In the duration of first 20 seconds, after the detected signal became stable, the infusion of physiological saline was applied slowly. On the about 50th second the signal declined and its profile was similar to the modeling curve of FIG. 5. The declining rate of the signal can be proportional to the leaky rate of the infusion.

In summary, the invention declares a method, a device and a system in the application of infusion leakage detection, infusion interruption and remote monitoring for IV therapy. The second optical window applied on biological tissue allows that the wavelengths of the light source within it can be minimally absorbed by melanin of skin, oxygenized and deoxygenized hemoglobin in blood, also it can be absorbed greatly by the infusion liquid (water is the majority of the liquid) (see

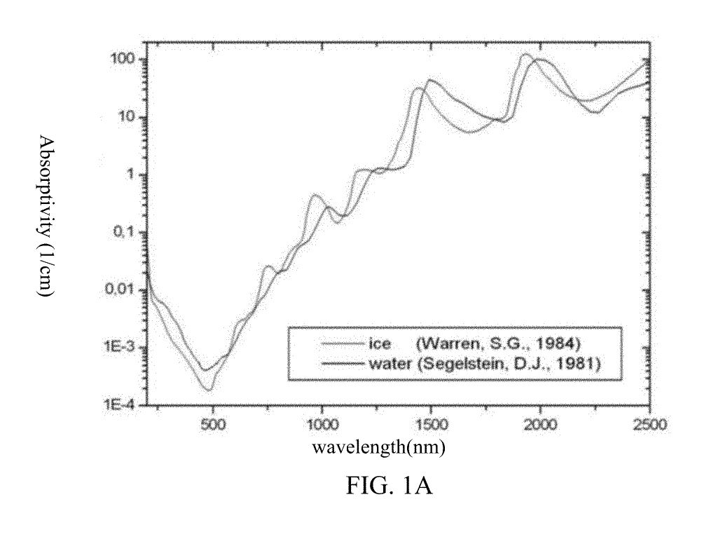

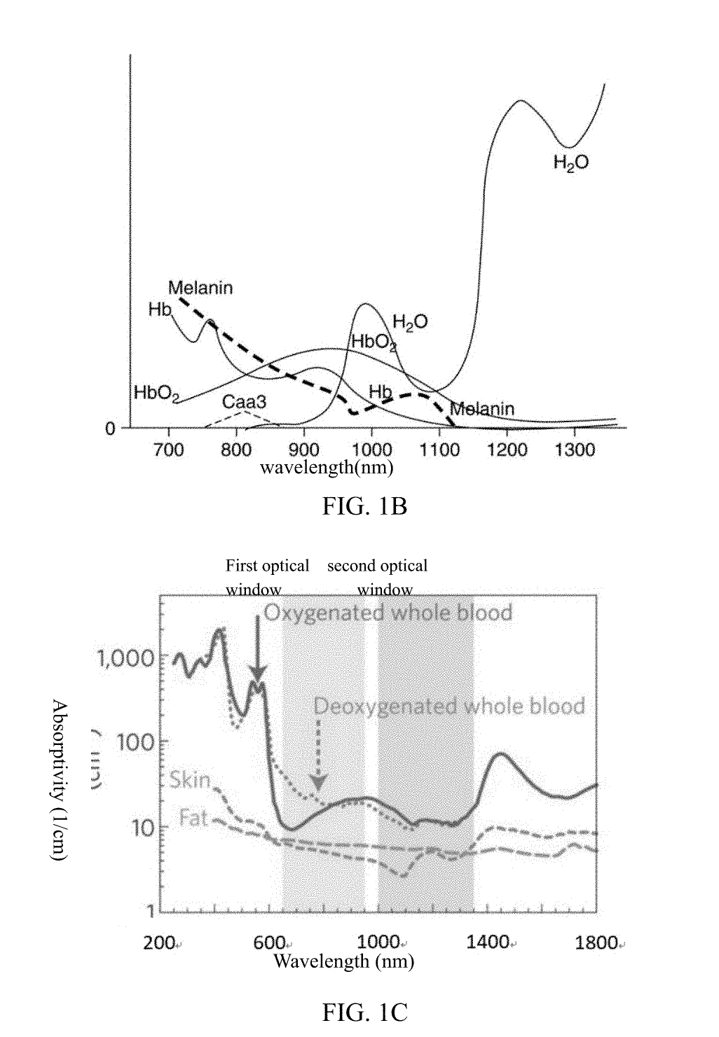

FIGS. 1A & 1B) when compared to the selected wavelengths within the first optical window (see FIG. 1C). The selection of the proper wavelengths accompanied with the characters of the second biological optical window can make the detecting device more sensitive to the occurrence of infusion leakage. In addition, the movement (acceleration) detector provides an additional information. The error-correction mechanism of the algorithm of this invention can ensure that the device can have more accurate leakage detection. Other than that, the remote monitoring system can save the physicians/nurses more time to take care of other services and no need to frequently take look the infusion situation of patients. An additional advantage of the system is the wireless and the detecting device which can be small enough to wear on the pasted on the body.

Although the present application has been explained above, it is not the limitation of the range, the sequence in practice, the material in practice, or the method in practice. Any modification or decoration for the present application is not detached from the spirit and the range of such.

What is claimed is:

1. An infusion-leakage detection device comprising:
a substrate;
a circuit with an infusion-leakage detection region formed on the substrate, the region can be aligned to an IV catheter which is inserted into a blood vessel of a patient, and the region comprises at least a light-emitting element and at least a light detector, wherein the light-emitting element emits a light with wavelength within the second optical window of biological organization to a target organization of a human body, and the light detector receives the light reflected, transmitted, diffused or scattered from the target organization to generate an electrical signal; and
a circuit and battery region also formed on the substrate, comprising a control and calculation unit, and an acceleration detector which is connected to the control and calculation unit;
wherein the control and calculation unit connects to the light-emitting element to control the light intensity of the light-emitting element;
wherein the acceleration detector senses body movement of a patient and provides a three dimensional acceleration signals of the movement to the control and calculation unit, and the control and calculation unit judges whether the body movement influences the actual infusion signal.

2. The infusion-leakage detection device of claim 1, further comprising: an amplifier formed on the substrate, configured to amplify the signal from the light detector and feed this signal to the control and calculation unit for further data processing and leakage/no-leakage judgement; a first communication unit formed on the substrate, configured to bridge the control and calculation unit and a remote monitoring equipment; and an alarm unit formed on the substrate, configured to receive an alarm command from the control and calculation unit to generate an alarm signal; wherein if the control and calculation unit determines or judges the input signal from the amplifier as leakage, then the alarm command is sent to the alarm unit and the first communication unit, respectively, to generate the alarm signals such as flashing light from an LED and tweet from a buzzer on the infusion-leakage detection device and to the remote monitoring equipment comprising a smart phone/tablet, or a computer of the nurse station through a wireless network.

3. The infusion-leakage detection device of claim 1, further comprising an indicating region formed on the substrate, the indicating region comprises an open groove on one side of the infusion-leakage detection device to hemi-surround the hub at the proximal end of the IV catheter, and a region which is a semi-transparent or transparent area with some width along a central line of the detection device for seeing through.

4. The infusion-leakage detection device of claim 3, further comprising a line or arrow marker on the central line of the indicating region configured to align the infusion-leakage detection region to the IV catheter and its hub.

5. The infusion-leakage detection device of claim 1, wherein the wavelength range of the light-emitting element is within or covers the second biological optical window which is about from 1000 nm to 1350 nm.

6. The infusion-leakage detection device of claim 1, wherein the light emitted from the light-emitting element can be a DC light or an AC light.

7. The infusion-leakage detection device of claim 1, wherein there have one of the light-emitting element and two of the light detectors, and the light-emitting element and the light detectors are arranged in a form of triangle.

8. The infusion-leakage detection device of claim 1, wherein there have two of the light-emitting elements and two of the light detectors, both the light-emitting elements and the light detectors are arranged in a form of rectangular or square, and the two light-emitting elements are located diagonally and same to the light detectors.

9. The infusion-leakage detection device of claim 1, wherein the substrate comprises a flexible printed circuit (FPC) or a printed circuit board (PCB).

10. an infusion-leakage detection and interruption system, comprising:
an infusion-leakage detection device of claim 2;
a server; and
an infusion interruption device:
wherein the infusion-leakage detection device transfers the detected data and alarm signal to the server and the infusion interruption device via a wireless network;
wherein the server connects to an intranet network system in a hospital so that the detected infusion-leakage data and warning signal can be delivered to the monitoring computer of the nurse station or the smart phone/tablet;
wherein the infusion-leakage interruption device is activated when receiving the signal of alarm command from the infusion-leakage detection device to block the infusion flow in an infusion conduit connected to the IV catheter to cease the leakage not to get worse.

11. The infusion-leakage detection and interruption system of claim 10, wherein the infusion-leakage interruption device comprises: a control unit; a second communication unit configured to receive an alarm command signal transmitted from the infusion-leakage detection device or from the wireless network; and a pinch valve having a fixed holder in which the infusion conduit is placed; wherein when the second communication unit receives an alarm command signal, it triggers the control unit to control the pinch valve to pinch off the infusion conduit.

12. The infusion-leakage detection and interruption system of claim 10, wherein the infusion-leakage detection device further comprises an indicating region formed on the substrate, the indicating region comprises an open groove on one side of the infusion-leakage detection device to hemi-surround the hub at the proximal end of, and a region which is a semi-transparent or transparent area with some width along the central line for seeing through.

13. The infusion-leakage detection and interruption system of claim 10, further comprising a line or arrow marker on the central line of the indicating region configured to align the infusion-leakage detection region to the IV catheter and its hub.

14. The infusion-leakage detection and interruption system of claim 10, wherein the wavelength range of the emitting light of the light-emitting element is within or covers the second biological optical window which is about from 1000 nm to 1350 nm.

15. The infusion-leakage detection and interruption system of claim 10, wherein the light emitted from the light-emitting element can be a DC light or an AC light.

16. The infusion-leakage detection and interruption system of claim 10, wherein there have one of the light-emitting element and two of the light detectors, and the light-emitting element and the light detectors are arranged in a form of triangle.

17. The infusion-leakage detection and interruption system of claim 10, wherein there have two of the light-emitting elements and two of the light detectors, and the light-emitting elements and the light detectors are arranged in a form of rectangular or square, and the two light-emitting elements are located diagonally and same to the light detectors.

18. The infusion-leakage detection and interruption system of claim 10, wherein the substrate comprises a flexible printed circuit (FPC) or a printed circuit board (PCB).
